(19) United States
(12) Patent Application Publication
(10) Pub. No.: US 2006/0217282 A1
(43) Pub. Date: Sep. 28, 2006

(54) Prion decontamination

(76) Inventors: Graham Stuart Jackson, London (GB); John Collinge, London (GB); Anthony R Clarke, London (GB)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/532,265

(22) PCT Filed: Oct. 29, 2003
(86) PCT No.: PCT/GB03/04651

§ 371 Date: Mar. 22, 2006

Related U.S. Application Data
(60) Provisional application No. 60/484,391, filed on Jul. 2, 2003.

(30) Foreign Application Priority Data
Nov. 1, 2002 (GB) 0225553.7

(51) Int. Cl. C11D 3/386

(52) U.S. Cl. 510/320

(57) ABSTRACT

The present invention relates to methods for prion decontamination comprising (i) contacting an entity to be decontaminated with a detergent, (ii) contacting said entity with one or more protease(s), and optionally (iii) autoclaving said entity. The invention further relates to kits comprising detergents and proteases for prion decontamination, and compositions comprising detergents and proteases for prion decontamination.

### FIELD OF THE INVENTION

The present invention relates to methods and reagents for use in prion decontamination. In particular, the invention relates to prion decontamination of surgical instnuments.

### BACKGROUND TO THE INVENTION

The persistence and resistance of the prion agents responsible for vCJD has raised fears about the possibility of iatrogenic transmission following surgery.

The prion diseases, which include scrapie in sheep, BSE in cattle and CJD in humans are a novel group of transmissible, fatal neurodegenerative conditions. The transmissible agent termed a prion is comprised largely or solely of a conformational isomer of a normal cellular PrP protein. This conformer designated PrPSc, has several unusual properties including resistance to proteolysis, detergent insolubility and high thermal stability. These physical properties coupled to recent observations that PrP^{Sc} adheres strongly to surgical steel present problems in the cleaning and sterilisation of surgical instruments as prion infectivity is known to be resistant to conventional autoclaving.

In the absence of a pre-clinical diagnostic test for vCJD pre-surgical testing of patients is not possible. Although in a minority of cases where CJD is suspected or confirmed used instruments can be quarantined or destroyed, for the majority of procedures new methods of decontamination are required. The UK Department of Health is currently engaged in several research projects in order to try to address the problem of iatrogenic CJD transmissions.

Standard autoclaving, and in some cases high temperature autoclaving to 1340C, is the hospital standard for prion decontamination. However, conventional studies have shown survival of prions under autoclave conditions. Clearly prions will gradually accumulate under these conditions. Furthermore, prions which survive heat treatment can become more resistant to decontamination and so highly resistant prions can accumulate on surgical instruments subjected to repeat autoclaving without prion destruction. Moreover, even the most effective autoclave will only sterilise to the degree that heat and steam penetrate the articles being treated. This is not straighforward when dealing with surgical sets comprising numerous complex instruments.

The WHO guidelines on prion decontamination recommend autoclaving immersed in 1 MNaOH. This is an extremely hazardous procedure. Furthermore, in addition to the safety aspects, the corrosive effect of such caustic alkali at that concentration, combined with the temperatures and pressures implicit to autoclaving, would be likely to destroy or at least seriously damage delicate surgical instruments.

Commercial reagents currently in use for cleaning of surgical instruments prior to autoclaving have little or no effect upon PrPSc contamination.

Existing methods of decontamination such as those involving LpH, LpHse, and Endozyme Plus are of limited use in destroying infectivity.

Taylor 1999 (Taylor D. M., Inactivation of prions by physical and chemical means. 1999, J. Hosp. Infect. 43, 69-76.) discloses the use of sodium hypochlorite solutions and 2M sodium hydroxide in prion inactivation. However, there are problems with this approach such as incomplete inactivation. Furthermore, resistance of prions to autoclaving is reported.

The present invention seeks to overcome problem(s) associated with the prior art.

### SUMMARY OF THE INVENTION

The inventors have developed a combined detergent and proteolytic enzyme treatment that reduces the titre of prion infectivity. In some embodiments reduction by about 1 million fold is achieved. The reagents used are water soluble, stable and of low toxicity. The protocol for their use is compatible with existing hardware such as machines used for pre-washing medical instruments prior to autoclaving. Thus the invention provides methods and reagents by which entities such as medical instruments can be decontaminated of prion infectivity.

The methods of the present invention, in particular the combination methods, are effective for prion inactivation such as vCJD prion inactivation. The methods find particular application on surfaces such as metal surfaces.

The methods and compositions of the present invention such as reagents for addition to hospital instrument washing machines find application in hospital or sterile services providers throughout the world to reduce the possibility of prion contamination such as that leading to iatrogenic CJD.

Advantageously, the methods of the present invention are used in addition to, and preferably preceding, autoclaving.

It is surprisingly shown herein that vCJD prions are relatively thermolabile compared to other prions. This surprising finding allows enhancement of the methods of the present invention as described below.

Without wishing to be bound by theory, the present invention makes use of specific knowledge of PrP^{Sc} chemistry. The inventors formulated the rationale that treating (eg. boiling) with detergent such as SDS should render PrP^{Sc} more susceptible to proteases. Numerous proteases and conditions were then examined which further led to the invention of the methods of the present invention.

### DETAILED DESCRIPTION OF THE INVENTION

In one aspect, the present invention provides a method for prion decontamination by enhancing the destructive effect of autoclaving. In this aspect, the present invention provides a method comprising (i) contacting an entity to be decontaminated with. a detergent such as SDS, and (ii) autoclaving said entity.

In another aspect, the invention provides a method for prion decontamination comprising (i) contacting an entity to be decontaminated with a detergent, (ii) contacting said entity with a protease, and optionally (iii) autoclaving said entity.

In another aspect, the invention provides a method for prion decontamination comprising (i) contacting an entity to be decontaminated with a detergent, (ii) contacting said entity with a first protease, (iii) contacting said entity with a second protease, and optionally (iv) autoclaving said entity.

Preferably said first and second proteases are different The entity may be contacted with the first and second proteases simultaneously or sequentially. Preferably the entity is contacted with the first and second proteases sequentially.

Optionally, further protease treatment steps may be incorporated in the methods of the present invention. Thus, in another aspect, the invention provides a method for prion decontamination comprising (i) contacting an entity to be decontaminated with a detergent, (ii) contacting said entity with a first protease, (iii) contacting said entity with a second protease, (iv) contacting said entity with one or more further proteases, and optionally (v) autoclaving said entity.

Proteases

Temperature/protease concentration optima:

As is plain to a person of ordinary skill in the art, the higher the concentration of protease(s), the greater and more rapid destruction is achieved. Combinations of protease concentration and time may be chosen according to need. These can be optimised by routine trial and error.

Examples presented herein include conditions optimal for use in automated washing machines. Furthermore, the conditions chosen are advantageously low in cost.

Incubation temperatures for the protease varies according to the protease used. Generally, any temperature from room temperature (eg. 20° C.) up to 60° C. is acceptable. The preferred temperature for papain is 30° C., the preferred temperature for ProteinaseK is 55° C., the preferred temperature for pronase is 45° C., the preferred temperature for Bromelain is 40° C. As the temperature moves away from the optimum for a particular protease, deactivation takes longer. Clearly, this can be compensated for by incubating for a longer time or using a greater concentration of protease. At temperatures above 60° C. activities can be lower and the enzymes can become inactivated, but clearly individual protease preparations will have individual deactivation temperatures and the manufacturers' guidance should be followed wherever possible.

Proteases can be adversely affected (eg. suffer reduced activity or loss of activity) in the presence of excess detergent. Individual proteases have individual characteristics, and it is well within the abilities of a person skilled in the art to avoid loss of activity due to detergent action. Manufacturers' guidance should be followed wherever possible. Advantageously detergent level(s) are reduced so as not to significantly inhibit protease activity before/at the time of contact with protease.

The protease is preferably selected from a group consisting of protease and peptidase enzymes belonging to class E.C. 3.4.-.- as defined by the Nomenclature Committee of the International Union of Biochemistry and Molecular Biology (http://www.chem.qmw.ac.uk/iubmb/enzyme/).

The protease can be mixture of proteases. However, it should be noted that when contacting with several proteases at once, individual activities can be reduced and compensation might be necessary eg. by longer time of contact. This is discussed in more detail below.

Preferably the protease comprises one or more of ProteinaseK, Pronase, Papain, or Bromelain.

When the protease is bromelain, preferably detergent is substantially absent at the protease step.

Where at least two protease steps are used, preferably at least one of the proteases comprises ProteinaseK.

When only a single protease step is used, preferably the protease comprises ProteinaseK, pronase or papain, more preferably ProteinaseK or pronase, more preferably ProteinaseK.

Preferably at least two protease steps are used.

Preferably at least one detergent step and at least two protease steps are used.

Proteases are susceptible to genetic and/or peptide or chemical level manipulation or modification. It will be apparent to a person skilled in the art that truncations, mutations or adaptation of the proteases (eg. to make them more protease resistant themselves) does not interfere with the invention provided that the peptidase activity of the enzyme(s) is retained by such manipulation(s). Indeed, it is accepted that pronase is more in the nature of a fractionated protease preparation rather than a recombinantly purified enzyme, and use of a sub-fractionation product of pronase or of a cloned and recombinantly purified fraction of the peptidase(s) comprised by pronase are embraced by the present invention. Thermostable proteases are particularly preferred, whether obtained by modification of existing proteases or by cloning proteases from thermophilic organisms.

In one aspect the invention provides a method for prion decontamination comprising (i) contacting an entity to be decontaminated with a detergent, (ii) contacting said entity with pronase, and optionally (iii) autoclaving said entity.

In a preferred aspect the invention provides a method for prion decontamination comprising (i) contacting an entity to be decontaminated with a detergent, (ii) contacting said entity with ProteinaseK, and optionally (iii) autoclaving said entity.

In a preferred aspect the invention provides a method for prion decontamination comprising (i) contacting an entity to be decontaminated with a detergent, (ii) contacting said entity with pronase, (iii) contacting said entity with papain, and optionally (iv) autoclaving said entity.

In a preferred aspect the invention provides a method for prion decontamination comprising (i) contacting an entity to be decontaminated with a detergent, (ii) contacting said entity with ProteinaseK, (iii) contacting said entity with pronase, and optionally (iv) autoclaving said entity.

In a preferred aspect the invention provides a method for prion decontamination comprising (i) contacting an entity to be decontaminated with a detergent, (ii) contacting said entity with pronase, (iii) contacting said entity with ProteinaseK, and optionally (iv) autoclaving said entity.

Sequential/Simultaneous Contact

Where more than one protease is used, the proteases may be combined into a single step. However, protease activity can be lowered in such an embodiment due to each protease digesting the other. Thus, the individual steps in the methods of the present invention are advantageously carried out sequentially for optimum efficacy. Furthermore, advantageously at least a proportion of the first protease is removed before the entity is contacted with the second or further protease. More preferably substantially all of the first protease is removed before contact with the second or further protease. This applies equally to each protease step in a multi-step sequence.

Detergent step(s) and protease step(s) should advantageously not be combined as the detergent may inactivate the protease(s). Thus, advantageously at least a proportion of the detergent is removed (or diluted) before the entity is contacted with the protease in order to maximise protease activity. Preferably the detergent step(s) precede protease step(s).

Detergent

The detergent is preferably an ionic detergent, preferably an anionic detergent, preferably one or more of Sodium dodecyl sulphate (SDS), Sodium taurodeoxycholate hydrate, Sodium 1-octanesulfonate monohydrate, Lithium dodecyl sulfate or N-Lauroylsarcosine sodium salt. Preferably the detergent is SDS.

Detergent may be used at any effective concentration. This may be easily determined and/or optimised by trial and error. When the detergent is SDS, the final concentration of the detergent with regard to the contacting with a detergent step is preferably about 2% to about 6%, preferably about 3% to about 5%, preferably about 4%, preferably 4%.

The entity may be contacted with the detergent at any suitable temperature. The optimal temperature for the detergent step is flexible and is preferably at least 70° C., preferably at least 80° C., preferably at least 90° C., preferably at least 100° C. The temperature may be constrained by the nature of the entity, for example some medical equipment such as endoscopes cannot tolerate high temperatures such as those used in autoclaving. For these situations, the methods of the invention advantageously do not involve autoclave conditions, and the temperature choice should be made by the operator with regard to the tolerances of the entity being decontaminated. Examples of methods according to the present invention which avoid the use of autoclave conditions may be found in protocol B in the Examples section. Advantageously methods according to the present invention such as those found in protocol B in the

Examples section may replace conventional prior art treatments such as LpH, LpHse, and EndozymePlus treatment.

The time of incubation for the detergent step is flexible and is preferably at least 2 minutes. Protracted incubation at the detergent step can be advantageous, such as hours or days or even longer.

Autoclaving

Autoclaving can be carried out following any suitable autoclave cycle. Typical cycles include 121° C. for 18 minutes or preferably 134° C. for 18 minutes. Alternative cycles may be chosen by the operator to suit their particular needs. Extended autoclave cycles may be advantageously employed.

Advantageously an autoclaving step is performed as a final step in the methods of the present invention. Combining the methods of the present invention with an autoclaving step has the further advantage of minimising spread of infection via the entity being decontaminated such as surgical instruments. Furthermore, by combining with autoclaving in this manner, there may advantageously be a multiplicative increase in efficacy, ie. if each method can reduce infectious titre by 5 logs then combining them may reduce infectivity by even more, such as by 10 logs.

Entity to be Decontaminated

The entity to be decontaminated may be any physical item for which it is desired to deactivate or remove prions. The term embraces solution(s) as well as objects such as devices or medical instruments (including surgical instruments), particularly metal objects or part(s) thereof. The prions to be deactivated or removed may be in the entity (eg. in solution or suspension), or may be on the entity (eg. bound, attached or otherwise associated with a surface of the entity). Thus, the entity may comprise a surface. Said surface may be a surface of a medical instrument. Said surface may comprise metal. Said metal may be steel such as surgical steel.

Decontamination

Decontamination refers to reduction in prion titre in a specific sample or setting. Decontanination may refer to the removal of prions firom a surface whether or not said prions are deactivated. Thus, decontamination includes deactivation and also includes the elimination of prions without regard to whether or not they are destroyedldeactivated. When decontaminating, it is important that prion infectivity is removed from the surface or solution being decontaminated. This may be by destruction (deactivation) or by simple separation. The important aspect is that prions (ie. PrP^{Sc}) are no longer associated with the surface or solution being decontaminated or are reduced in number and/or titre. Clearly, if non-infective prion fragments remain adhered to the surface after decontamination, this would not materially affect the decontamination or the fact that said surface had been successfully decontaminated.

Decontamination may be assessed by any suitable assay. Preferably, the assay used is western blotting or bioassay. Clearly assays such as bioassays and/or western blotting assays have a sensitivity limit. So long as prion titre (prion number/infectivity) has been reduced, then prion decontamination will be considered to have taken place.

Preferably prion decontamination is 100 fold, preferably 1000 fold, preferably 10,000 fold, preferably 100,000 fold, preferably 1,000,000 fold or even more. Preferably prions are completely eliminated or deactivated.

Assay Methods

The reduction in prions produced by the methods of the present invention may be monitored by any suitable means known in the art. Specific examples of suitable assay techniques are provided herein to illustrate the assessment of prion reduction. Clearly, certain methods will present themselves as more suitable for a given situation than other methods. For example, if prion decontamination is taling place in solution, then a western blotting approach might be most suitable. If prion decontamination is taking place on a surface, then direct visualisation on that surface might be most suitable. Alternatively for prion decontamination taking place on a surface, bioassay might be the most suitable. Choice of individual assay methods for individual situations is well within the capabilities of a person skilled in the art. It will be appreciated that in many situations the most important indicator is loss/reduction of infectivity. Currently, prion infectivity is most usually assessed by bioassay. However, biochemical assay of the infective conformer PrPsc is equally appropriate.

An example of a suitable monitoring method is an immunoblotting assay. Advantageously the immunoblotting assay is, or is based on, the assay described in Wadsworth et al 2001 Lancet vol 358 pp 171-180.

An example of a suitable monitoring method is a bioassay. Bioassay methods are generally geared towards the individual prion species being assayed. Selection of suitable bioassay methods is advantageously based on the prion species being assayed.

Kits

The present invention also relates to kits for use in decontamination. Thus the invention provides a kit comprising detergent and a protease selected from the group consisting of ProteinaseK, papain, pronase, and bromelain. Preferably the kit comprises two or more such proteases. Preferably the detergent in the kit is SDS.

Compositions

The present invention also relates to compositions for use in decontamination. In one aspect, the invention provides a composition comprising an ionic detergent and one or more proteases selected from the group consisting of ProteinaseK, papain, pronase, and broinelain. In a preferred aspect, the composition comprises two or more such proteases. In a more preferred aspect, the detergent of the composition is SDS.

The invention is now illustrated by way of examples which should not be regarded as limiting in scope. In the Examples, reference is made to the following figures:

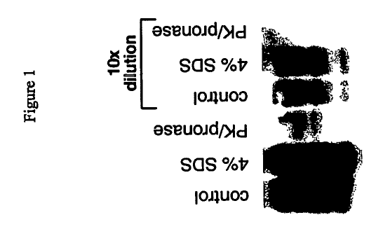

FIG. 1 shows a western blot.

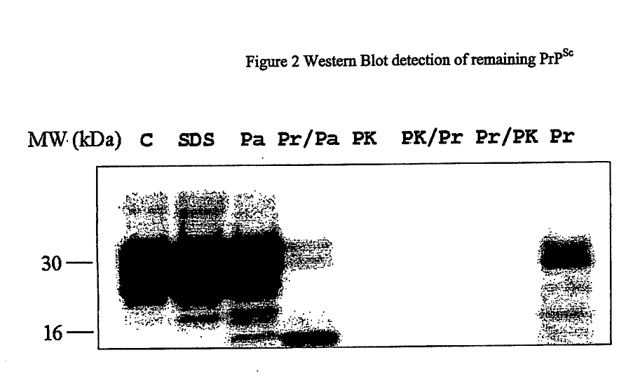

FIG. 2 shows a western blot.

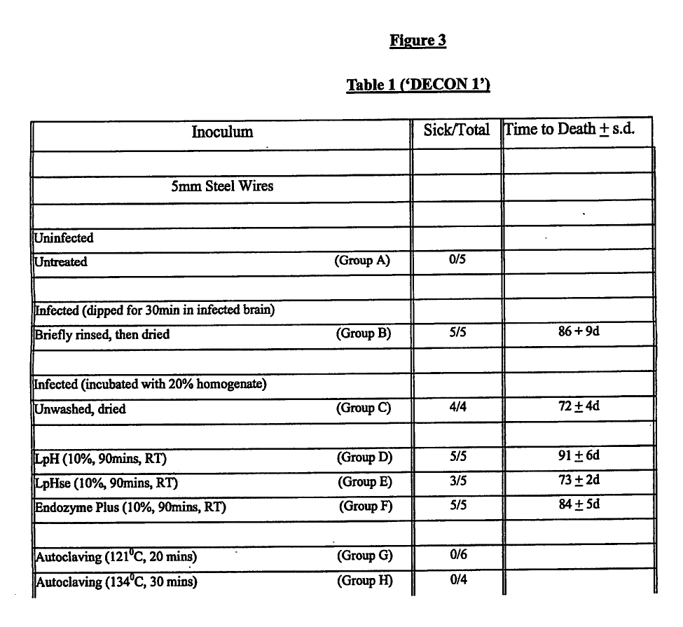

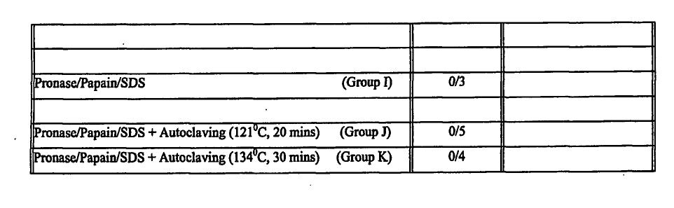

FIG. 3 shows a table.

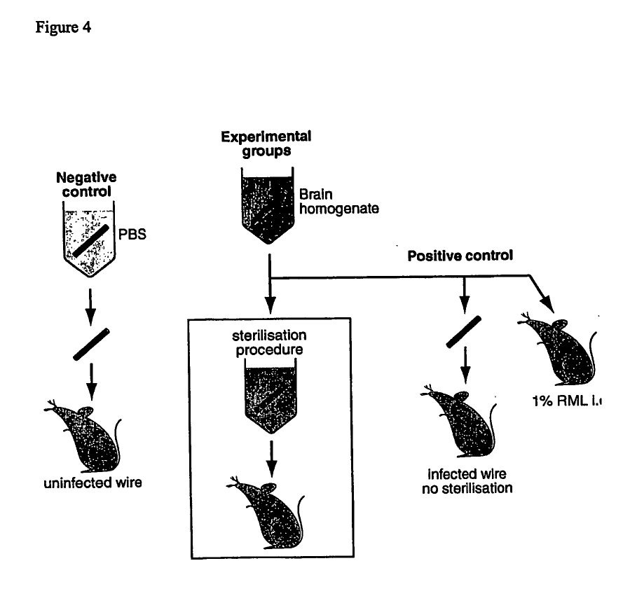

FIG. 4 shows a diagram of experimental design.

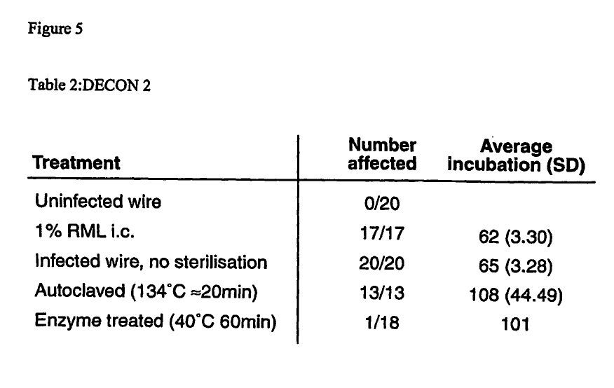

FIG. 5 shows a table.

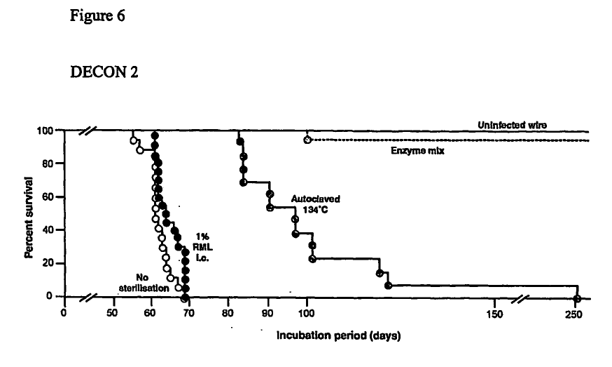

FIG. 6 shows a graph.

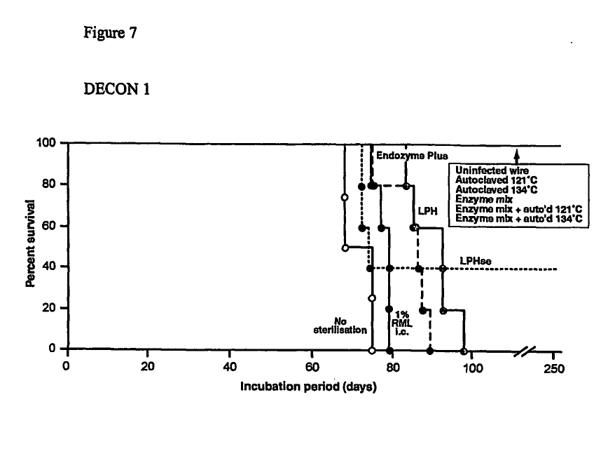

FIG. 7 shows a graph.

### EXAMPLE 1

Combined Detergent and Protease Treatment

A combined detergent and proteolytic enzyme treatment that reduces the titre of prion infectivity by up to a million fold is demonstrated.

This estimate is based upon a previously determined detection limit for the specific Western Blotting protocol used (Wadsworth et al Lancet 2001) which is a preferred assay method.

Using this assay method, it was determined that we can detect 5 nl of a 10% w/v brain homogenate following PK digestion. In FIG. 1, each of the three lanes represents a sodium phosphotungstate precipitated pellet from 10 ml of a 10% w/v brain homogenate. It can be seen that after treatment according to protocol A that PrP is only just visible ie about 5 nl equivalent. This initially contained 10,000,000 nl equivalents so the level of destruction is around 1 million fold

The reagents used are water soluble, stable and of low toxicity. The protocol for their use is compatible with existing hardware for example as used in hospital decontamination departments for pre-washing and autoclaving instruments. Thus the invention provides for decontamination of prion infectivity from surgical instruments. Advantageously the methods of the present invention can be implemented using existing machinery.

### EXAMPLE 2

Destruction of PrPSc in Aqueous Samples

This example describes methods by which the infectious material PrP^{Sc} can be deactivated in an aqueous suspension by serial exposure of the entity (in the example the entity is infected brain tissue) to two proteolytic enzymes (ProteinaseK and pronase or pronase and papain) in the presence of a detergent.

The level of deactivation (in this example deactivation correlates with destruction) can be estimated from immunodetection of Western blots.

Preparation of Tissue Sample

Brain of vCJD-infected human frontal cortex was homogenised to 20% w/v in PBS Dulbecco's (GIBCO-BRL 14190-094) by passing the brain tissue through 18-gauge, 21-gauge and 23-gauge needles. The homogenate was diluted to 15% w/v with PBS, frozen in small aliquots and stored at −70° C.

PROTOCOL A: Three-Phase Destruction of PrP^{Sc} From vCJD Brain Homogenate Using ProteinaseK and Pronase.

(a) Treatment with detergent: sodium dodecyl sulphate (SDS). A 20 μl aliquot of 15% homogenate was measured into a screw-capped Eppendorf tube and 5 μl of 20% SDS was added to give a final concentration of 4% SDS. The mixture was heated for 15 minutes at 99-100° C. and then cooled to room temperature. Cooling to 40° C. would be equally acceptable.

(b) Treatment with a first protease: ProteinaseK (PK). A solution of 40 μg/ml PK was prepared in double distilled water. A 5 μl aliquot of this enzyme solution was added to the 25 μl solution of detergent-treated homogenate produced by procedure (a), above. i.e. Final conc of ProteinaseK is 6.6 ug/ml-The mixture was incubated at 40° C. for 30 minutes.

(c) Treatment with a second protease: pronase. A solution of 2 mg/ml pronase was made up in 0.1 M TRIS/HCl pH 7.5. A 0.5 μl aliquot of this enzyme solution was added to the 30 μl solution of SDS- and PK-treated homogenate produced by serial procedures (a) and (b), above. i.e. Final conc of pronase is 33 ug/ml. The mixture was incubated at 40° C. for 30 minutes.

PROTOCOL B: Three-Phase Destruction of PrP^{Sc} From vCJD Brain Homogenate Using Pronase and Papain.

(a) Treatment with detergent: sodium dodecyl sulphate (SDS). A 20 Pi aliquot of 15% homogenate was measured into a screw-capped Eppendorf tube and 5 μl of 20% SDS was added to give a final concentration of 4% SDS. The mixture was heated for 15 minutes at 99-100° C. And then cooled to between RT and 40° C. before proceeding to next step.

(b) Treatment with a first protease: pronase. A solution of 0.5 mg/ml pronase was made up in 0.1 M TRIS/HCl pH 7.5. A 5 μl aliquot of this enzyme solution was added to the 25 μl solution of detergent-treated homogenate produced by procedure (a), above. I.e. Final conc of pronase is 83 ug/ml. The mixture was incubated at 40° C. for 30 minutes.

(c) Treatment with a second protease: papain. A solution of 0.4 mg/ml papain was made up in 0.1 M TRIS/HCl pH 6.0. A 0.5 μl aliquot of this enzyme solution was added to the 30 μl solution of SDS- and pronase-treated homogenate produced by serial procedures (a) and (b), above. Final conc of papain is 6.5 ug/ml. The mixture was incubated at 40° C. for 30 minutes.

Detection of PrP^{Sc} by Western Blot

The materials from the SDS/PK/pronase (Protocol A) and the SDS/pronase/papain (Protocol B) treatments described above were submitted to Western blot analysis, see FIG. 2. The blots were visualized using the antibodies ICSM 18 and ICSM 35 to detect any remaining PrP^{Sc} in the samples. Using either antibody, there was no detectable PrP^{Sc}.

Following various enzymatic treatments detailed above the samples were subjected to SDS-PAGE and visualised by Western blotting and detection with the antibody ICSM35. The first lane (Lane 1—untreated material) is a control sample of untreated material.

The second lane is treated with just 4% w/v SDS at 100° C. for 15 minutes (Lane 2—4% SDS 100° C. 15 mins).

The third lane shows SDS and papain treatment, (Lane 3—4% SDS & papain: two steps corresponding to step 1 & step 3 in protocol B)

The fourth lane combines SDS pronase then papain (Lane 4—Protocol B ie three steps.)

The fifth lane is SDS followed by PK alone (Lane 5—the times, concentrations and temperatures are as steps 1 & 2 of Protocol A)

The sixth lane shows SDS and sequential PK then pronase—(Lane 6—Protocol A)

The seventh lane shows SDS and sequential pronase then PK—(Lane 7 Protocol A but with steps 2 & 3 reversed)

The final eighth lane is SDS and pronase alone (Lane 8—just steps 1 & 2 from protocol B).

Similar results are observed when ICSM18 is used for visualisation of the Westem blot.

A note on interpretation of FIG. 2: All the bands are PrP. The control lane contains total PrP, ie both PrP^{C} and PrP^{Sc}. It is not possible to define which are PrP^{Sc} α posterori as these are functionally defined. However, in the absence of any PrP we can then say that PrP^{Sc} is absent. It is clear from studying FIG. 2 that the methods of the present invention lead to significant prion decontamination. For example, with Protocol B (Lane 4) there are some immunoreactive bands remaining. These are PrP, (most likely to be PrP^{Sc}) however, it is plain that the level has been drastically reduced from the starting material, thereby demonstrating significant prion decontamination according to the present invention.

### EXAMPLE 3

Destruction of Prion Infectivity on Surgical Steel

In this example, the application of the methods of the present invention to the destruction of prion infectivity on surfaces (in this example the surfaces are surgical steel), so demonstrating the applicability to the decontamination of surgical instruments. Again, advantageously the methods of the invention may be implemented in pre-washing procedures carried out in hospital decontamination departments. Small samples of this surgical steel are implanted into transgenic mice to bioassay for residual infectivity.

This experiment was designed to demonstrate the efficacy of disinfection provided by enzymatic treatment compared to existing treatment methods. 5 mm×0.15 mm steel wires were incubated for 30 minutes with a 20% homogenate prepared from the brain of a CD1 mouse terminally sick with Rocky Mountain Laboratories (RML) scrapie. These wires were then dried without washing and inserted into the brains of Tg20/ZH1 transgenic mice (which had been bred to overexpress the normal prion protein PrP^{c}).

Before insertion wires were either not subjected to disinfection (positive control group), incubated for 90 minutes at RT with existing disinfection compounds (LpH, LpHse, Endozyme Plus), autoclaved or treated with enzymes (with or without autoclaving).

The enzymatic treatment comprised heating at 99-100° C. in 4% SDS for 15 minutes, followed by cooling to 40° C. followed by the addition of pronase followed by papain.

Each enzymatic incubation was performed at 40° C. for 30 minutes. (i.e. protocol B) (Protocol A (SDS, PK, Pronase) is also suitable for decontaminating surgical instruments—see Example 4 (‘DECON 2’))

The wires were then briefly rinsed with PBS. A negative control group was provided by inserting wires that had not been incubated with RML homogenate.

To confirm the infectivity (ie. the capacity to induce clinical disease) of the RML homogenate in which the wires were incubated, 30 μl of different dilutions including a 1% dilution were inoculated intra-cerebrally into further groups of mice. This confirmed that the homogenate used was infectious. By comparison of the different dilutions, the infectious titre of the homogenate may be quantitatively determined if required.

In order to test the effect of exposing steel to intact brain (as opposed to a 20% homogenate) one group of mice had wires inserted that had been dipped for 30 minutes in the brain of a terminally sick CD1 mouse. These wires were briefly rinsed in Phosphate Buffered Saline—Dulbecco's (PBS) and then dried before insertion.

Detailed Experimental Procedures:

Materials

Pronase (SIGMA CODE P5147; 1.16 mg/ml in 0.1 M TRIS/HCl pH 7.5)

Papain (SIGMA CODE P3375; 0.21 mg/ml in 0.1 M TRIS/HCl pH 6.0)

Implantation of Uninfected Wire

Single segments of untreated wires were manually implanted into the brain of Tg20 mice by using a G26 needle as guide.

(Group A)

Dipping and Implantation of Dipped Wires

Ten wire segments (5 mm) were transiently inserted into the brain of a RML-infected CD1 mouse (right side of bregma) for 30 min. “Dipped” wire segments were briefly rinsed in 50 ml PBS Dulbecco's (GIBCO-BRL 14190-094) using a 50 ml FALCON tube and dried for 30 min. on a petri dish at room temperature. A single segment was manually implanted into the brain of each of six mice by using a G26 needle as guide. The remaing four wire segments took no fither part in this example.

(Group B)

Preparation of 20% (w/v) RML Brain Homogenate:

Brain homogenate (20% w/v) was prepared in PBS Dulbecco's (GIBCO-BRL 14190-094) by passing the brain through 18-g, 21-g and 23 gauge needles. Brain of RML-infected CD1 mouse used for dipping (wet weight: 400 mg) was homogenized in 1 ml PBS. Sample was adjusted to a final volume of 2 ml with PBS. The total homogenate called 20% (w/v) (without any centrifugation step) and used immediately for the next step. Aliquots were frozen at −20° C.

Exposure of Wire to 20% (w/v) RML Brain Homogenate:

Groups of 20 wire segments were exposed to freshly prepared 20% w/v RML brain homogenate (0.1 ml) in a 1.5 ml Safe4ock Eppendorf tube and incubated for 30 min at 22° C. with agitation. After incubation, brain homogenate was drawn off by a pipette and the exposed wires transferred directly to a petri dish. Wires were dried for 30 min at room temperature. Single segments were manually implanted into the brain of each mouse by using a G26 needle as guide.

(Group C)

Treatments for Decontamination:

Treatments with LpH, LpHse and Endozyme Plus

LPH/LPHse (Steris, Steris House, Jays Close, Viables, Basingstoke, Hants RG22 4AX UK), Endozyme Plus (The Ruhof Corporation: 393 Sagamore Avenue: Mineola, N.Y. 11501). LPH and LPHse are proprietary compounds designed for disinfection of worktops and similar surfaces that may be exposed to contamination. Endozyme Plus is marketed for disinfection of medical instruments.

Three groups of twenty wire segments exposed to brain homogenate (as described above) and transferred into a Eppendorf tube containing 0.2 ml of solution (LpH, LpHse, or Endozyme Plus; each at 10% v,v in double distilled water. They were incubated for 90 min at room temperature. (Solutions were prepared prior to use by addition of 0.15 ml stock to 1.35 ml ddwater). Wires were briefly rinsed with 50 ml and 25 ml PBS.

LpH (Group D)

LpHse (Group E)

Endozyme Plus (Group F)

Autoclaving

Twenty wire segments exposed to brain homogenate (see 3) were put in a sealed autoclave bag and autoclaved on an uncovered autoclave tray at 121° C. for 20 min and at 134° C. for 30 min, respectively. Autoclaved wire segments were implanted into the brains of Tg20 mice (single segment each).

121° C. (Group G)

134° C. (Group H)

Enzymatic Treatment Alone or Followed by Autoclaving

Twenty wire segments exposed to brain homogenate (see 3) were transferred into an Eppendorf tube containing 4% w/v SDS in double distilled water and boiled at 100° C. for 15 min. Eppendorf tubes were cooled down to 40° C. in the presence of 4% SDS and liquid was removed by pipetting. 100 μl of pronase solution (1.16 mg/ml) was added to the same Eppendorf tube and incubated at 40° C. for 30 min. Solution was removed and 100 μl of papain solution (0.21 mg/ml) was added to the same Eppendorf tube and further incubation at 40° C. for 30 min. Wires were briefly rinsed with 50 ml and 25 ml PBS. One group of treated wires was assayed for infectivity by permanent implantation of a single segment into the brain of Tg20 mouse

(Group I)

Two groups of such treated wires were subjected to autoclaving in parallel to the sample as descnbed in 4.1. and later a single segment implanted into the brain of each Tg20 transgenic mouse.

Enzymatic treatment and 121° C. 20 min (Group J)

Enzymatic treatment and 134° C. 20 min (Group K)

Results are shown in table 1 (see FIG. 3: ‘DECON 1’)). Time course of the results may be seen in FIG. 7 (‘DECON 1’).

It is thereby demonstrated that the methods of the present invention result in significant prion decontamination.

### EXAMPLE 4

Destruction of Prion Infectivity on Surgical Steel

In this example, the application of the methods of the present invention to the destruction of prion infectivity on surfaces (in this example the surfaces are surgical steel), so demonstrating the applicability to the decontamination of surgical instruments. Again, advantageously the methods of the invention may be implemented in pre-washing procedures carried out in hospital decontamination departments. Small samples of this surgical steel are implanted into transgenic mice to bioassay for residual infectivity.

This experiment was designed to demonstrate the efficacy of disinfection provided by enzymatic treatment compared to existing treatment methods. 5 mm×0.15 mm steel wires were incubated for 30 minutes with a 10% homogenate prepared from the brain of a CD1 mouse terminally sick with Rocky Mountain Laboratories (RML) scrapie. These wires were then briefly washed in 500 ul PBS on a vortex mixer to remove loosely adherent tissue fragments, dried, and inserted into the brains of Tg20/ZH1 transgenic mice (which had been bred to overexpress the normal prion protein PrP^{c}).

Before insertion wires were either not subjected to disinfection (positive control group ‘Infected wire, no sterilisation’), autoclaved at 134° C., or treated with enzymes according to the invention as described in this example. The individual treatments are summarised in FIG. 5 (Table 2).

The enzymatic treatment was carried out according to Protocol A of example 2 (SDS, PK, Pronase); the wire was incubated at 99-100° C. in 4% SDS for 15 minutes, and cooling to 40° C., followed by the addition of ProteinaseK, followed by addition of pronase. Each enzymatic incubation was performed at 40° C. for 30 mins.

The wires were then dried. A negative control group was provided by inserting wires that had not been incubated with RML homogenate.

To measure the infectivity of the RML homogenate in which the wires were incubated, 30 ul of a 1% dilution was inoculated intra-cerebrally into a further group of mice. This confirmed that the homogenate used was infectious.

Detailed Experimental Procedures:

Materials

Proteinase K (MERCK CODE 390973P; 0.00625 mg/ml in 0.1 M TRISIHC1 pH 7.6) Pronase (SIGMA CODE P5147; 0.0336 mg/ml in 0.1 M TRIS/HCl pH 7.6)

Implantation of Wires

Single segments of treatedluntreated wires were manually implanted into the brain of Tg20 mice by using a G26 needle as guide.

Preparation of 10% (w/v) RML Brain Homogenate:

Brain homogenate (10% w/v) was prepared essentially as described above, i.e. prepared in PBS by passing the brain through sequentially smaller bore needles (18-g, 21-g and 23 gauge needles). Aliquots were fiozen at −70° C.

Other procedures were as described above.

Each of the groups of treated wires were implanted into the brains of indicator mice and the numbers of animals assayed and the results are shown in FIG. 5 (Table 2).

The time course of the assay is described in the graphs of FIG. 6 (‘DECON 2’).

As can be seen, the uninfected wire led to no disease. The i.c. infected material gave 100% disease, as did the untreated infected wire segments.

The high temperature autoclave treatment which is the standard hospital treatment for prion decontamination also gave 100% disease.

Finally, the treatment according to the present invention resulted in only one animal from 18 becoming diseased. At this level, the affected animal in this group was probably only exposed to a single infective dose.

Therefore, it can be seen that prion decontamination of entities carried out according to the present invention results in extremely effective reduction of infectivity. This reduction is to a level which can virtually be regarded as prion free.

All publications mentioned in the above specification are herein incorporated by reference. Various modifications and variations of the described methods and compositions of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for prion decontamination of an entity comprising (i) contacting said entity with a detergent, (ii) contacting said entity with a protease, and optionally (iii) autoclaving said entity.

2. A method according to claim I wherein the protease is selected from a group consisting of protease and peptidase enzymes belonging to class E. C. 3.4.-.- as defined by the Nomenclature Committee of the International Union of Biochemistry and Molecular Biology.

3. A method according to claim 1 wherein the protease is selected from the group consisting of ProteinaseK, papain, pronase, and bromelain.

4. A method according to claim 3 wherein the protease is pronase.

5. A method according to claim 3 wherein the protease is ProteinaseK.

6. A method for prion decontamination comprising (i) contacting an entity to be decontaminated with a detergent, (ii) contacting said entity with a first protease, (iii) contacting said entity with a second protease, and optionally (iv) autoclaving said entity.

7. A method according to claim 6 wherein the first and second proteases are selected from the group consisting of ProteinaseK, papain, pronase, and bromelain.

8. A method according to claim 7 wherein the first protease is pronase and the second protease is papain.

9. A method according to claim 7 wherein the first protease is ProteinaseK and the second protease is pronase.

10. A method according to claim 7 wherein the first protease is pronase and the second protease is ProteinaseK.

11. A method according to claim 1 wherein the detergent is an ionic detergent.

12. A method according to claim 11 wherein the detergent is an anionic detergent.

13. A method according to claim 12 wherein the detergent is SDS.

14. A method according to claim 1 wherein said entity comprises a surface.

15. A method according to claim 14 wherein said surface is a surface of a medical instrument.

16. A method according to claim 14 wherein said surface comprises metal.

17. A method according to claim 16 wherein said metal is steel.

18. A kit comprising detergent and a protease selected from the group consisting of ProteinaseK, papain, pronase, and bromelain.

19. A kit according to claim 18 comprising two or more said proteases.

20. A kit according to claim 18 wherein said detergent is SDS

21. A composition comprising an ionic detergent and one or more proteases selected from the group consisting of ProteinaseK, papain, pronase, and bromelain.

22. A composition according to claim 21 comprising two or more said proteases.

23. A composition according to claim 21 wherein said detergent is SDS.

24. A method according to claim 6 wherein the detergent is an ionic detergent.

25. A method according to claim 15 wherein said surface comprises metal.

26. A kit according to claim 19 wherein said detergent is SDS

27. A composition according to claim 22 wherein said detergent is SDS.
